(19) United States
(12) Patent Application Publication
(10) Pub. No.: US 2015/0031994 A1
(43) Pub. Date: Jan. 29, 2015

(54) Method Of Fitting A User With An Athletic Mouthguard

(71) Applicant: Christopher L. Straatmann, Chesterfield, MO (US)

(72) Inventor: Christopher L. Straatmann, Chesterfield, MO (US)

(21) Appl. No.: 13/948,714

(22) Filed: Jul. 23, 2013

(51) Int. Cl. A63B 71/08; A61B 6/00; A61B 6/03; A61B 5/00; A61B 8/08

(52) U.S. Cl. CPC A63B 71/085; A61B 5/4542; A61B 8/0875; A61B 6/032; A61B 6/505; A61B 5/4836; A61B 5/0059
USPC 600/427; 600/590; 600/439; 600/407; 600/476

(57) ABSTRACT

A method of fitting a user with an athletic mouthguard. The method comprises determining a position of a user's jaw to achieve desirable inhalation characteristics, and providing a mouthguard to the user. The mouthguard is of a size and shape to position the user's jaw in approximately the determined desired position. The mouthguard is shaped and configured to enhance performance of the user during an athletic endeavor.

### CROSS-REFERENCE TO RELATED APPLICATIONS

Not Applicable.

### STATEMENT REGARDING FEDERALLY SPONSORED RESEARCH OR DEVELOPMENT

Not Applicable

### APPENDIX

Not Applicable.

### BACKGROUND OF THE INVENTION

1. Field of the Invention

This invention pertains to fitting a user with an athletic mouthguard.

2. General Background

It is known to use a mouthguard to protect a user against injury during an athletic activity. But a mouthguard typically reduces an athlete's ability to breath, thereby reducing the athlete's performance.

### SUMMARY OF THE INVENTION

One aspect of the invention is a method of fitting a user with an athletic mouthguard. The method comprises determining a position of a user's jaw to achieve desirable inhalation characteristics, and providing a mouthguard to the user. The mouthguard is of a size and shape to position the user's jaw in approximately the determined desired position. The mouthguard is shaped and configured to enhance performance of the user during an athletic endeavor.

Another aspect of the invention is a method of fitting a user with an athletic mouthguard. The method comprises determining a position of a user's jaw to achieve desirable inhalation characteristics. The desirable inhalation characteristics are associated with an athletic activity. The method further comprises providing a mouthguard to the user. The mouthguard is of a size and shape to position the user's jaw in approximately the determined desired position.

Further features and advantages of the present invention, as well as the operation of the invention, are described in detail below with reference to the accompanying drawings.

### BRIEF DESCRIPTION OF THE DRAWINGS

FIG. 1 is a top rear isometric view of a first embodiment of an athletic mouthguard in accordance with the present invention;

FIG. 2 is a bottom rear isometric view of the athletic mouthguard of FIG. 1;

FIG. 3 is a side-elevational view of the athletic mouthguard of FIGS. 1 and 2;

FIG. 4 is a cross-sectional view taken along the plane of line 4-4 of FIG. 3;

FIG. 5 is a top rear isometric view of a second embodiment of an athletic mouthguard in accordance with the present invention; and

FIG. 6 is a bottom rear isometric view of a third embodiment of an athletic mouthguard in accordance with the present invention.

Reference numerals in the written specification and in the drawing figures indicate corresponding items.

### DETAILED DESCRIPTION

The fitting of a user with an athletic mouthguard in accordance with a preferred method of the present invention comprises first determining a position of a user's jaw to achieve desirable inhalation characteristics, and then providing a mouthguard to the user in which the mouthguard is of a size and shape to position the user's jaw in approximately the determined desired position. The mouthguard is shaped and configured to enhance performance of the user during an athletic endeavor.

Preferably, the desirable inhalation characteristics are associated with an athletic activity. The desirable inhalation characteristics for an athletic activity may be different depending on the anaerobic and aerobic components of the athletic activities. Some athletic activities are more anaerobic and less aerobic. Some activities are less anaerobic and more aerobic. For example, American football is typically more anaerobic and less aerobic than soccer. The position of a user's jaw to achieve desirable inhalation characteristics may include determining a position of a user's jaw which provides at least near the maximum pharyngeal airway size of the user during inhalation by the user. In accordance with the present embodiment, determining a position of a user's jaw which provides at least near the maximum pharyngeal airway size of the user during inhalation by the user may be accomplished at least in part by one or more of the following: acoustic reflection, computerized tomography, and fluoroscopy. The step of determining a position of a user's jaw which provides at least near the maximum pharyngeal airway size of the user during inhalation by the user may be accomplished at least in part with a pharyngometer. The step of determining a position of a user's jaw which provides at least near the maximum pharyngeal airway size of the user during inhalation by the user may be accomplished at least in part by digital imaging.

During the step of determining a position of a user's jaw to achieve desirable inhalation characteristics, the user's jaw is preferably moved to a plurality of positions with readings made during inhalation by the user at each jaw position. The user's jaw may be moved generally vertically to vary the vertical spacing between the user's upper teeth and lower teeth. The user's jaw may also be moved horizontally (preferably forward and back) to vary the horizontal orientation of the lower teeth relative to the upper teeth. The position of the user's jaw relative to the user's upper teeth which provides the desirable inhalation characteristics associated with the athletic activity is noted, and then a mouthguard is provided to the user in which the mouthguard is of a size and shape to position the user's jaw in approximately the determined desired position.

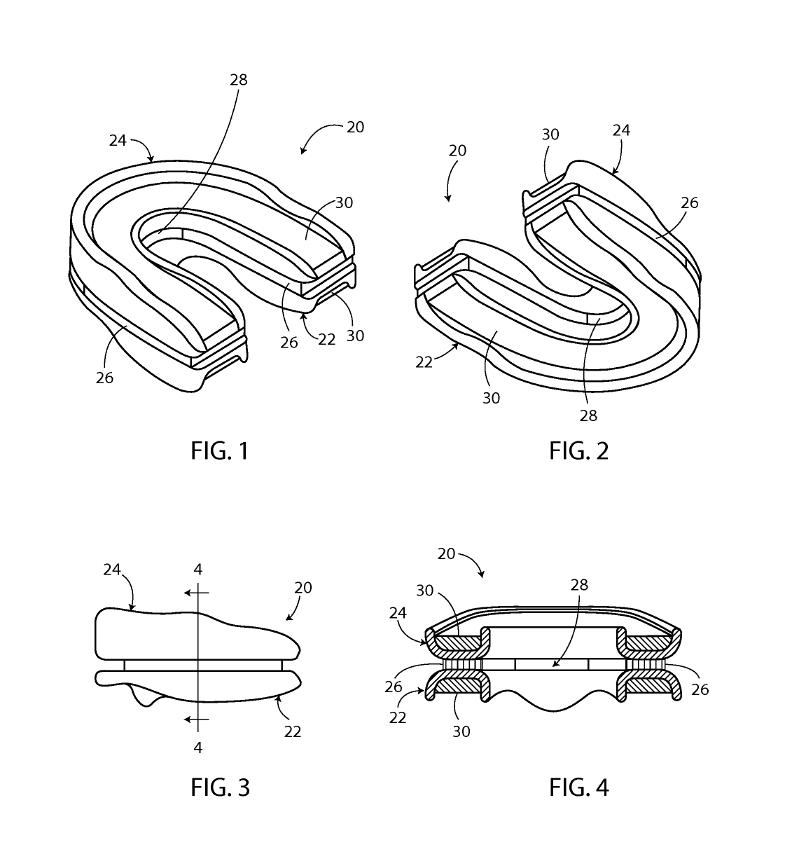

A first embodiment of an athletic mouthguard in accordance with the present invention is generally indicated by the reference numeral 20 in FIGS. 1-4. The mouthguard 20 comprises a base portion, generally indicated at 22, an upper portion, generally indicated at 24, and two spacer portions 26. The base portion 22 is adapted and configured to be engaged by the lower teeth of a user. The upper portion 24 is adapted and configured to be engaged by the upper teeth of a user. The spacer portions 26 operatively connect the base portion 22 and the upper portion 24. The spacer portions 26 also vertically spaces the upper portion 24 from the base portion 22 such that when the user wears the mouthguard 20, the user's upper teeth are spaced from the user's lower teeth a predetermined amount. The spacer portions 26 further horizontally fix the position of the base portion 22 relative to the upper portion 24 such that when the user wears the mouthguard 20, the mouthguard positions the user's lower teeth and jaw in a desired horizontal position relative to the user's upper teeth. In this embodiment, the base portion 22 is generally U-shaped and the upper portion is generally U-shaped. In this embodiment, the base portion 22, upper portion 24 and spacer portions 26 define an air gap 28 to facilitate breathing by the user wearing the mouthguard 20. The base portion 22, upper portion 24 and spacer portions 26 may be individual pieces secured together, such as by a suitable adhesive or because of a mechanical interfit. Alternatively, the base portion 22, upper portion 24 and spacer portions 26 may all constitute portions of a single, unitary construction. The base portion 22, upper portion 24 and spacer portions 26 may be of a suitable polymeric material, such as ethyl vinyl acetate. Alternatively, the spacer portions 26 may be of a material different from that of the base portion 22 and/or the upper portion 24. Preferably, the cavity of the base portion 22 and the cavity of the upper portion 24 include a filler 30. Preferably, the material of the filler 30 has a lower melting temperature than the material of the main components of the base portion 22 and the upper portion 24. For example, the material of the filler 30 may be of a lower melt grade of EVA than that of the other components such that the filler softens upon immersion of the mouthguard 20 in boiling water. As such the mouthguard 20 is a boil and bite type of mouthguard in which the filler 30 is conformable to the shape of the teeth of the user. Other material that may be used include Kraton styrene polymer material, PVC, and ENGAGE™, a polyethylene polymer product produced by Dow Chemical.

The combined thicknesses of the base portion 22, upper portion 24 and spacer portions 26 and the orientation of the base portion 22 relative to the upper portion 24 are selected such that the mouthguard 20, when being properly worn by the user, positions the user's jaw in approximately the determined desired position. Also, the size and shape of the air gap 28 may be selected to enhance the athletic performance of the user.

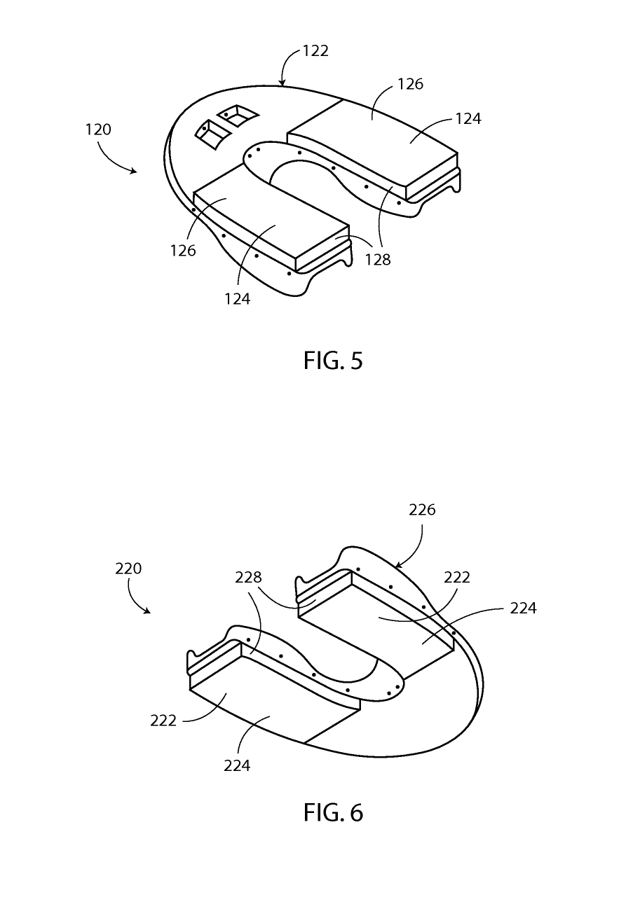

Referring now to FIG. 5, another embodiment of an athletic mouthguard in accordance with the present invention is generally indicated by the reference numeral 120. The mouthguard 120 is similar to the mouthguard 20 of FIGS. 1-4, except the mouthguard 120 has spacer elements 124 projecting upward from a U-shaped base portion 122. Each spacer element 124 comprises an upper portion 126 and a spacer portion 128. The base portion 122 is adapted and configured to be engaged by the lower teeth of a user. The upper portions 126 are adapted and configured to be engaged by the upper teeth of a user. The spacer portions 128 operatively connect the base portion 22 and the two upper portions 126. The mouthguard 120 is adapted to accommodate a user who prefers a mouthguard that does not surround the user's upper teeth. Although not shown, it is to be understood that the cavity of the base portion 122 may be filled with a filler similar to the filler 30 of the embodiment of FIGS. 1-4. The mouthguard 120 also includes apertures through which a strap may be secured to tether the mouthguard to a helmet. Although not shown in the other embodiments, it is to be understood that the other embodiments may also include strap receiving apertures.

Referring now to FIG. 6, another embodiment of an athletic mouthguard in accordance with the present invention is generally indicated by the reference numeral 220. The mouthguard 220 is similar to the mouthguard 120 of FIG. 5, except the mouthguard 220 has spacer elements 224 projecting downward from a single U-shaped upper portion 226. Each spacer element 224 comprises a base portion 222 and a spacer portion 228. The two base portions 222 are adapted and configured to be engaged by the lower teeth of a user. The upper portion 126 is adapted and configured to be engaged by the upper teeth of a user. The spacer portions 128 operatively connect the two base portions 222 and the upper portion 226. Although not shown, it is to be understood that the cavity of the upper portion 226 may be filled with a filler similar to the filler 30 of the embodiment of FIGS. 1-4. The mouthguard 220 is adapted to accommodate a user who prefers a mouthguard that does not surround the user's lower teeth.

In view of the foregoing, it should be appreciated that the invention has several advantages over the prior art.

As various modifications could be made in the constructions and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by any of the above-described exemplary embodiments, but should be defined only in accordance with the following claims appended hereto and their equivalents.

It should also be understood that when introducing elements of the present invention in the claims or in the above description of exemplary embodiments of the invention, the terms “comprising,” “including,” and “having” are intended to be open-ended and mean that there may be additional elements other than the listed elements. Additionally, the term “portion” should be construed as meaning some or all of the item or element that it qualifies. Moreover, use of identifiers such as first, second, and third should not be construed in a manner imposing any relative position or time sequence between limitations.

What is claimed is:

1. A method of fitting a user with an athletic mouthguard, the method comprising:
determining a position of a user's jaw to achieve desirable inhalation characteristics;
providing a mouthguard to the user, the mouthguard being of a size and shape to position the user's jaw in approximately the determined desired position, the mouthguard being shaped and configured to enhance performance of the user during an athletic endeavor.

2. A method as set forth in claim 1 wherein the determining the position of a user's jaw to achieve desirable inhalation characteristics comprises determining a position of a user's jaw which provides at least near the maximum pharyngeal airway size of the user during inhalation by the user.

3. A method as set forth in claim 2 wherein the determining a position of a user's jaw which provides at least near the maximum pharyngeal airway size of the user during inhalation by the user is accomplished at least in part by one or more of the following: acoustic reflection, computerized tomography, and fluoroscopy.

4. A method as set forth in claim 2 wherein the determining a position of a user's jaw which provides at least near the maximum pharyngeal airway size of the user during inhalation by the user is accomplished at least in part by acoustic reflection.

5. A method as set forth in claim 2 wherein the determining a position of a user's jaw which provides at least near the maximum pharyngeal airway size of the user during inhalation by the user is accomplished at least in part with a pharyngometer.

6. A method as set forth in claim 2 wherein the determining a position of a user's jaw which provides at least near the maximum pharyngeal airway size of the user during inhalation by the user is accomplished at least in part by digital imaging.

7. A method as set forth in claim 1 wherein the user's jaw is moved during the step of determining the position of a user's jaw.

8. A method as set forth in claim 1 wherein the mouthguard comprises a base portion, an upper portion and a spacer portion operatively connecting the base portion and the upper portion, the base portion of the mouthguard being adapted and configured to be engaged by lower teeth of the user, the upper portion of the mouthguard being adapted and configured to be engaged by upper teeth of the user.

9. A method as set forth in claim 8 wherein the base portion is generally U-shaped.

10. A method as set forth in claim 8 wherein the upper portion is generally U-shaped.

11. A method as set forth in claim 8 wherein the base portion is generally U-shaped and wherein the upper portion is generally U-shaped.

12. A method of fitting a user with an athletic mouthguard, the method comprising:
determining a position of a user's jaw to achieve desirable inhalation characteristics, the desirable inhalation characteristics being associated with an athletic activity;
providing a mouthguard to the user, the mouthguard being of a size and shape to position the user's jaw in approximately the determined desired position.

13. A method as set forth in claim 12 wherein the determining the position of a user's jaw to achieve desirable inhalation characteristics comprises determining a position of a user's jaw which provides at least near the maximum pharyngeal airway size of the user during inhalation by the user.

14. A method as set forth in claim 13 wherein the determining a position of a user's jaw which provides at least near the maximum pharyngeal airway size of the user during inhalation by the user is accomplished at least in part with a pharyngometer.

15. A method as set forth in claim 14 wherein the mouthguard comprises a base portion, an upper portion and a spacer portion operatively connecting the base portion and the upper portion, the base portion of the mouthguard being adapted and configured to be engaged by lower teeth of the user, the upper portion of the mouthguard being adapted and configured to be engaged by upper teeth of the user.
